(19) United States
(12) Patent Application Publication
(10) Pub. No.: US 2021/0299396 A1
(43) Pub. Date: Sep. 30, 2021

(54) BIPHASIC BREATHING VENTILATOR

(71) Applicants: Gopi Mohan, Newton, MA (US); Ryan Carroll, Boston, MA (US); David Griggs, Boston, MA (US)

(72) Inventors: Gopi Mohan, Newton, MA (US); Ryan Carroll, Boston, MA (US); David Griggs, Boston, MA (US)

(21) Appl. No.: 17/217,241

(22) Filed: Mar. 30, 2021

Related U.S. Application Data
(60) Provisional application No. 63/002,059, filed on Mar. 30, 2020.
(60) Provisional application No. 63/033,543, filed on Jun. 2, 2020.

(51) Int. Cl. A61M 16/20; A61M 16/10

(52) U.S. Cl. CPC A61M 16/209; A61M 16/1075; A61M 2205/3368; A61M 16/205; A61M 16/204

(57) ABSTRACT

A biphasic system and method for assisting breathing of a patient by controllably providing breathing gas to the patient and taking exhaled gas from the patient during inspiration and expiration phases. The system and method having a pair of pressure-controlling means such as hydrostatic pressures or pop-off valves that are coupled to the patient breathing interface. Various means for controlling gas pressure at the breathing interface are described.

### RELATED APPLICATIONS

This application claims the benefit of and priority to U.S. Provisional Application No. 63/002,059, filed on Mar. 30, 2020, and No. 63/033,543, filed on Jun. 2, 2020, both of which are hereby incorporated by reference.

### TECHNICAL FIELD

The present application relates to ventilators used to assist breathing.

### BACKGROUND

Patients who are unable to independently breathe or oxygenate the lungs on account of a number of conditions may require mechanical assistance. Premature newborn infants, those with chronic lung disease and others may require assisted breathing systems to survive. One type of system used to assist patients (especially very young babies) to breathe is a bubble CPAP (continuous positive air pressure) apparatus, which supplies air or oxygen gas to the patient and controls the pressure of said air or oxygen using a back-pressure provided by a controllable water column in a water tank as known to those skilled in the art.

Other types of mechanical and electronically controlled ventilator systems are available depending on the condition of the patient and the available medical facilities. However, as shown during certain acute medical emergencies such as the COVID-19 pandemic of 2020, there can become a shortage of emergency ventilators compared to the demand, which can be a life-threatening public health emergency at a local, national or international scale.

This invention may in some aspects provide solutions to assist breathing and oxygen delivery to patients, especially neonates. In other aspects the invention may alleviate shortages of ventilator machines where the present apparatus can be used instead of a ventilator machine so as to extend the available supply of ventilator machines to those requiring them. In yet other aspects the invention can offer solutions where the complexity, cost and availability of more expensive ventilator machines is prohibitive such as in field operations or in underdeveloped regions of the world.

This invention may also in some aspects avoid over-consumption of scarce resources such as breathing gases, which other examples of bubble ventilators can consume at excessive rates, e.g., 50 LPM, 70 LPM or more. Since the present invention can be deployed to under-resourced environments and locations, the rate of consumption of breathing gases during operation can be a concern, which is addressed below.

### SUMMARY

One aspect provides a biphasic breathing assistance system, comprising a breathing gas source comprising at least one gas to be used for breathing by a patient using said system; a gas flow network coupled to said breathing gas source; a patient breathing interface coupled to said gas flow network, configured and arranged to move an amount of the breathing gas to the patient during a first (inspiration) phase of operation and to take an amount of exhaled gas from the patient during a second (expiration) phase of operation; a first (PIP) gas line coupled to the gas flow network at a first end of the first (PIP) gas line and coupled to a first back-pressure regulator having a first back-pressure setting at a second end of the first (PIP) gas line; a second (PEEP) gas line coupled to the gas flow network at a first end of the second (PEEP) gas line and coupled to a second back-pressure regulator having a second back-pressure setting at a second end of the second (PEEP) gas line; a first gas isolation valve disposed in the gas flow network downstream of the breathing gas source and upstream of the patient breathing interface; a second gas isolation valve disposed in the gas flow network between said first (PIP) gas line and said second (PEEP) gas line; and wherein said first gas isolation valve is configured and arranged to be open during said first (inspiration) phase of operation of the system while the second gas isolation valve is shut, and the first gas isolation valve is configured and arranged to be shut during said second (expiration) phase of operation of the system while the second gas isolation valve is open.

Another aspect provides a method for assisting patient breathing using a biphasic breathing assistance apparatus, the method comprising providing a pressure-regulated breathing gas source to a supply side of a gas flow network; during an inspiration phase, opening a first gas isolation valve between said breathing gas source and a patient breathing interface coupled to said gas flow network to permit flow of breathing gas from said breathing gas source to said patient breathing interface, and closing a second gas isolation valve between said patient breathing interface and a gas exhaust side of said gas flow network; during an expiration phase, closing said first gas isolation valve and opening said second gas isolation valve so as to substantially block flow of breathing gas to the patient breathing network but to permit flow of exhaled gas from said patient breathing network to said gas exhaust side of the gas flow network.

Another aspect provides a system providing breathing gas to a patient, comprising a breathing gas source coupled to and in fluid communication with a gas flow network; a patient breathing interface in fluid communication with said gas flow network; a first liquid reservoir, containing a first volume of liquid, the first liquid reservoir in fluid communication with said gas flow network through a first gas tube having a first end thereof coupled to said gas flow network and a second end thereof submerged at a first depth defining a first liquid column height H1 and a corresponding first hydrostatic pressure HP1; a second liquid reservoir, containing a second volume of liquid, the second liquid reservoir in fluid communication with said gas flow network through a second gas tube having a first end thereof coupled to said gas flow network and a second end thereof submerged at a second depth defining a second liquid column height H2 and a corresponding second hydrostatic pressure HP2; a first gas isolation valve disposed in said gas flow network between the breathing gas source and the patient breathing interface, the first gas isolation valve having a first (open) state to permit flow of said breathing gas from the breathing gas source to the patient breathing interface during an inspiration phase of operation of the system, and a second (closed) state to block flow of said breathing gas from the breathing gas source to the patient breathing interface during an expiration phase of operation of the system; and a second gas isolation valve disposed in said gas flow network between the first ends of said first and second gas tubes, the second gas isolation valve having a first (open) state to permit flow of exhaled gas from the patient breathing interface to the second liquid reservoir during the expiration phase of operation of the system, and a second (closed) state to block flow of exhaled gas from said patient breathing interface to said second liquid reservoir.

### BRIEF DESCRIPTION OF THE DRAWINGS

For a fuller understanding of the nature and advantages of the present concepts, reference is made to the following detailed description of preferred embodiments and in connection with the accompanying drawings, in which:

FIG. 1 illustrates an exemplary system according to the present invention having a plurality of fluid chambers;

FIG. 2 illustrates another embodiment of the invention using a single liquid backpressure canister with two separate gas tubes;

FIG. 3a illustrates an embodiment during inspiration;

FIG. 3b illustrates the previous embodiment during expiration;

FIG. 4 illustrates an exemplary gas flow valve with sensor control;

FIG. 5 illustrates a manual operation of said gas flow valve;

FIG. 6 illustrates a control panel interface with indicators and alarm units;

FIG. 7 illustrates an embodiment using calibrated back pressure pop-off valves to control the gas pressure in the PIP and PEEP phases;

FIG. 8 illustrates an embodiment with a bypass line to provide continuous bias flow and avoid rebreathing;

FIG. 9 illustrates a compact valve block and manifold used in some embodiments;

FIG. 10 illustrates a topology for the gas flow network and components;

FIG. 11 illustrates a two-valve manifold;

FIG. 12 illustrates time sequences for PIP and PEEP along with relating opening and closing of Valve 1 and Valve 2 gas passages; and

FIG. 13 illustrates gas flow pathways during inspiration and expiration.

### DETAILED DESCRIPTION

A nasal CPAP may be used with distressed newborn infants in the neonatal intensive care unit, but the present disclosure may be applied to patients of any age, patients suffering a variety of conditions, and may be implemented in sophisticated clinical environments or in the field where full medical facilities are not available. This invention significantly reduces the rate of consumption and waste of precious breathing gas (e.g., bottled oxygen, air, and mixtures) using a biphasic breathing assistance system as described below. The invention can be provided in a compact and robust form factor and delivered relatively inexpensively for use in many treatment environments, clinics, field hospitals and other scenarios.

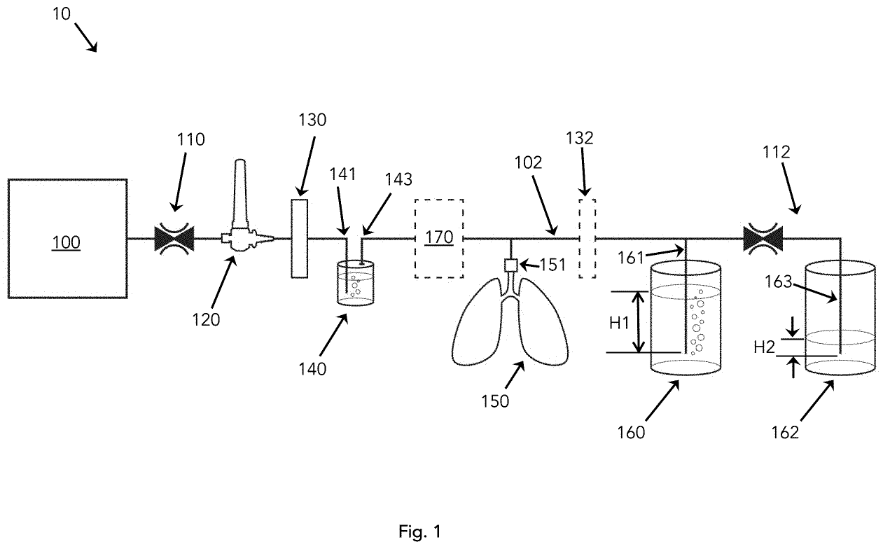

FIG. 1 illustrates an exemplary system 10 according to the present invention. The system receives one or more breathing gas sources 100 such as a clinical/hospital air or oxygen source, or compressed air or oxygen tank or other supply. A blender may be used to properly proportion a ratio of two breathing gases such as oxygen and air, but the use of a blender is not required in all embodiments.

Depending on the nature and pressure of the supply gas, a pressure regulator 120 may be employed to down-convert a high-pressure gas source to a lower pressure gas source for use in the system 10. Also, a filter 130 may be employed to remove unwanted particulates, microbes, contaminants, entrained oils or other objects from the breathing gas supply as warranted.

In a particular and non-limiting aspect, the system may include more than one filter. For example, a first filter 130 may be installed upstream in the patient inspiration pathway between the breathing gas source and the patient so as to filter unwanted particulates or contaminants, and a second filter 132 may be installed downstream in the patient expiration pathway so as to filter out dangerous things exhaled by the patient. By including a filter downstream of the patient (in the expiration path), the system can prevent exhaled droplets or pathogens from an ill patient from exiting into the clinical environment and infecting other patients or care givers or surfaces near the patient. For example, the second downstream filter 132 may comprise or be configured and arranged to meet one or more clinical recommendations (e.g., as a N95-based filter) that can trap COVID19 or other viruses, pathogens or infectious complications. The illustrated examples may not show filters, humidification chambers, sensors or other components described, but that is merely for simplification, and those skilled in the art will understand that these and other ancillary features can be installed as appropriate in a given situation.

In addition, a humidification chamber 140 may be employed (e.g., containing saline, water or other fluids) to control the humidity of the gas provided to the patient. In some embodiments, the humidification chamber 140 comprises a bubble chamber that contains a volume of liquid through which the breathing gas is forced to pass on its way to the patient 150 and other system components. For example, an inlet bubble tube or port 141 bubbles the unhumidified breathing gas into the liquid where it becomes humidified, and an outlet tube or port 143 allows the humidified breathing gas to exit the chamber.

Optionally, a temperature controller 170 may also be used to warm, cool or otherwise monitor or control a temperature of the breathing gas provided to the patient. For example, the temperature controller 170 may comprise a heat exchanger, an electric heating element, or a fluid heating and/or cooling element as appreciated by those skilled in the art. Temperature monitoring at one or more points in system 10 is also possible in some embodiments, including to control heating and/or cooling of the breathing gas supply to maintain a set temperature or to maintain gas temperature within a set temperature range.

The relative position of some of the components illustrated in the present examples is not limiting, as those skilled in the art would appreciate that different relative positions of some components can be implemented as appropriate in a given situation. For example, the relative location of the filter 130, humidification chamber 140, temperature regulator 170 and pressure regulator 120 or other components does not necessarily need to be in the configuration shown in the exemplary drawings.

Also, as stated one or more components described are not required for overall operation of the system 10 unless specified or claimed as such. Some components can be optional in various installations, e.g., to reduce cost or spatial footprint of the system 10. Specifically, some or all of the pressure regulator, filter, humidification chamber and temperature control means can be optional in situations where such auxiliary components are not needed or are not available (e.g., in extreme emergencies in under-equipped or field environments). The humidification chamber 140 is generally airtight and may itself be warmed to achieve or assist in temperature control of the breathing gas. Non-limiting flow rates may be between 0 and 100 liters per minute (LPM), however, as will be explained below, the invention is capable of operating at reduced breathing gas flow rates.

The breathing gas supply 100 (optionally blended, filtered, temperature- and pressure-regulated and humidified) is provided to a gas flow network 102 including a gas line or manifold such as a pipe or tube that connects the components of the system from the gas source to the patient and other components of the system. In some embodiments, the patient 150 receives the breathing gas through a suitable breathing assistance means, for example through a nasal tube or a face mask or other intubation means as known to those skilled in the art. In some instances, the patient wears a gas-tight or substantially gas-tight breathing mask providing pathways for inspiration (inhaling) and expiration (exhaling) of gases so that the gas pressure at the patient's mouth and/or nose are controllable, especially if the patient is not able to breathe on their own accord. The present system is therefore usable with a number of respiratory interfaces, including but not limited to endotracheal tubes, laryngeal masks, airway devices, full face BiPAP masks, or high-flow nasal cannula.

A plurality of backpressure regulating bubble canisters 160, 162 are coupled to the gas flow network 102. These canisters or liquid reservoirs include a first canister or reservoir 160 comprising a PIP (peak inspiratory pressure) regulating component filled with a first determined amount of liquid (e.g., water) wherein a first bubble tube 161 is submerged to a first given fluid column height so that a first height H1 of the liquid column above the lower opening/end of the bubble tube 161 in the PIP canister defines a first hydrostatic backpressure HP1 setting of the PIP canister 160. A second canister or reservoir 162 is also connected to the manifold or gas flow network 102, wherein the second canister 162 comprises a PEEP (positive end expiratory pressure) regulating component filled using a second determined amount of liquid (e.g., water) wherein a second bubble tube 163 is submerged to a second depth so that the height H2 of the liquid column above the lower opening/end of the bubble tube 163 in the PEEP canister 162 defines a second hydrostatic backpressure setting HP2 of the PEEP canister 162.

It can be seen that the first and second gas tubes 161, 163 submerged in their respective liquid columns in the respective liquid canisters or reservoirs 160, 162 have ends that couple the gas flow network 102 to each of the respective reservoirs 160, 162. So each such gas tube has a first end thereof in fluid communication with the gas flow network and a second end thereof open and terminating at some depth below the liquid surface within its respective canister.

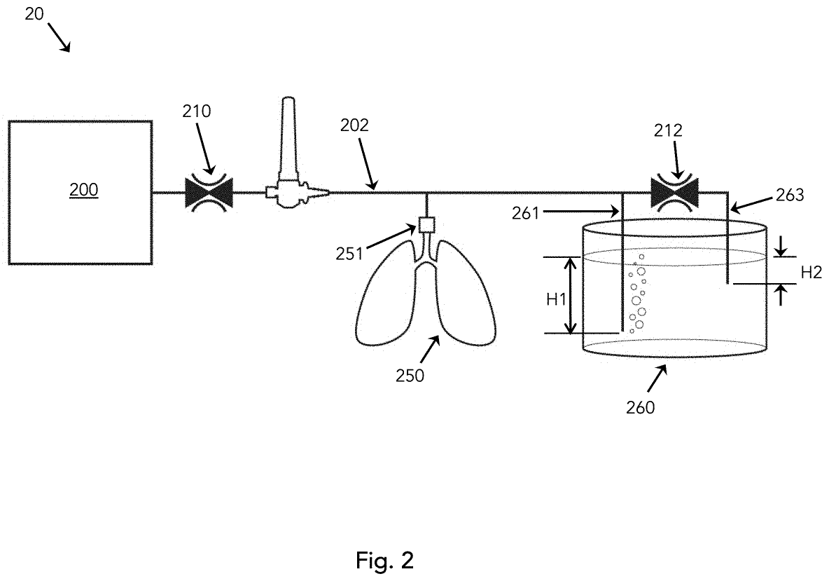

FIG. 2 illustrates an embodiment of a system 20 whereby the separate PIP and PEEP liquid backpressure canisters are replaced with a single liquid canister 260. Here, ends of separate gas bubble lines 261, 263 are submerged to different first and second depths H1, H2 below the liquid (e.g., water) line in the same canister unit 260. The PIP and PEEP gas tubes 261, 263 are submerged to respective depths H1, H2 below the surface of the fluid in the canister to establish the desired and adjustable water column height above the opening of the submerged tubes to establish the respective hydrostatic backpressure values HP1, HP2 therein. This single canister 260 arrangement can be used for most or all of the present examples as seen appropriate by those designing a given implementation of the invention. Also, it is noted that in this and some other examples a breathing gas source or gas blender 200 is represented at the gas source end of the system, but in a given embodiment this can be replaced or augmented with the other gas source and processing components described (e.g., air/oxygen source, filter(s), regulator(s), humidifier(s) and so on). The absence of one or more such components in an illustration is only for simplification and not by way of limitation. Similarly, an embodiment illustrated without ancillary components such as the filters, humidifiers or temperature controllers described above does not mean that the embodiment could not include those components if desirable.

As will be further described below, gas flow valves 110, 112, 210, 212 are disposed at one or more locations in the main gas flow lines 102, 202 to controllably permit or stop the flow of gas at certain locations in system 10, 20. Specifically, in some embodiments, separate valves 110, 210 are disposed upstream of the patient breathing connections 151, 251 and between the (PIP, PEEP) canister connections, respectively. First valve 110, 210 may be referred to as the inspiratory valve and the second valve 112, 212 may be referred to as the expiration valve for reasons that will be evident to those skilled in the art upon review of the present disclosure. The valves are operable and controllable so as to cause a necessary or desired sequence of gas flow through each of the two valves. Exemplary operation of the valves can be actuated by electrical, electronic, mechanical or other means, and a controller circuit may be used to actuate (open/shut) each of the inspiratory and expiratory valves according to a timing sequence which will be described herein.

It is noted that the hydrostatic pressures HP1, HP2 exerted by the PIP and PEEP liquid column heights are a function of the water columns HP1 and HP2 measured from its respective water surface to the opening at the bottom of the respective PIP/PEEP gas bubble tube ends (or the height of the submerged gas bubble tube portions). Generally, the hydrostatic backpressure HP1 of the (PIP) tube is greater than that of the (PEEP) tube HP2 for reasons that will are apparent upon review of the entirety of this disclosure. That is, the first gas bubble tube in the first (PIP) canister is generally submerged to a greater extent than the second gas bubble tube in the second (PEEP) canister, i.e., H1 is greater than H2 and HP1 is greater than HP2.

The liquid column height and corresponding hydrostatic pressure at the first bubble tube, wherein the first and second (PIP and PEEP) bubbler tubes are each coupled to the gas manifold 102, 202 but are separated by a controlled shutoff valve disposed in the gas manifold or flow network between the two tubes or canisters. The first (PIP) tube 161, 261 is coupled to the manifold on the same side of the shutoff valve 112, 212 as the patient 150, 250 on the upstream or supply side of the valve 112, 212 while the second (PEEP) tube is coupled to the manifold on the downstream side of the valve 112, 212.

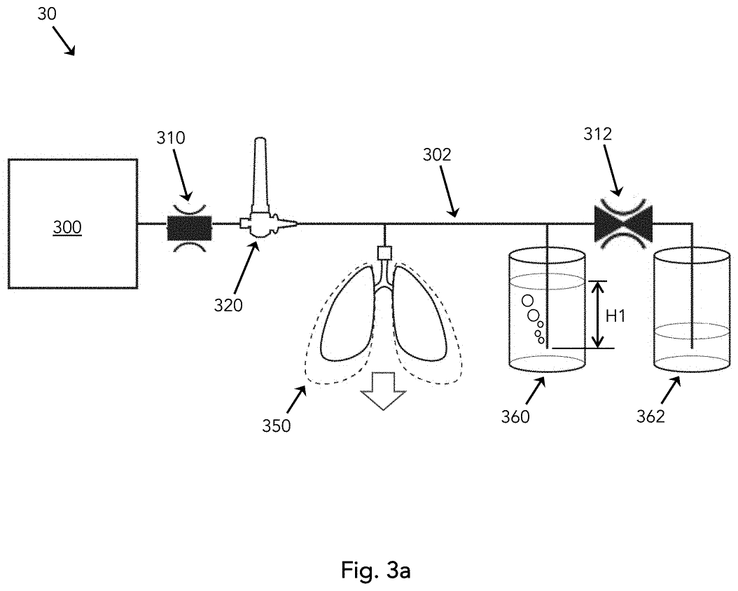

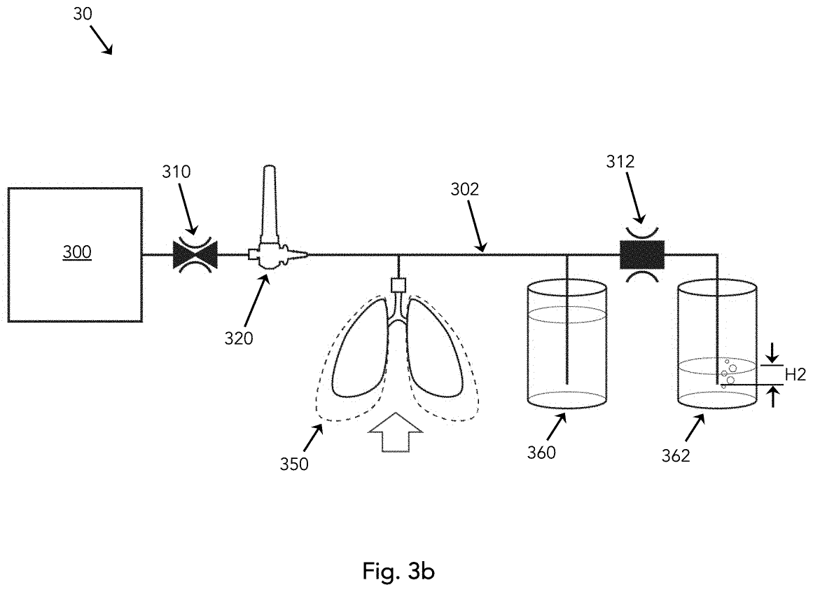

FIG. 3a illustrates a simplified exemplary system 30, shown configured during the inspiration (breath intake) phase. We see that the first valve (310 or the inspiratory or afferent valve sometimes referred to herein as Valve 1) is in the open position allowing gas flow therethrough while the second valve (312 or the expiratory or efferent valve, sometimes referred to herein as Valve 2) is in the closed position preventing gas flow therethrough. This causes the breathing gas 300 in the inspiration phase to pass through the PIP canister 360 at the backpressure determined by the height of the column of liquid H1 therein. That is, in the inspiratory phase, gas can flow from the gas source 300 to the patient 350 through the first (inspiratory) valve 310, but gas is prevented from flowing out through second (expiratory) valve 312.

FIG. 3b illustrates the system 30 in the expiratory phase (exhaling phase) of operation. The first (inspiratory) valve 310 is in the closed position blocking gas flow between the source 300 and patient 350 while the second (expiratory) valve 312 is in the open position. Excess gas and pressure above the second water column height H2 pressure in the second (PEEP) canister 362 will be ejected through the second gas bubble tube into the PEEP canister liquid which rises and escapes to the atmosphere. Here, no breathing gas flows from the source 300 to the patient 350 through valve 310 but gas can be expired from the patient 350 out through valve 312 and the PEEP canister 362, which is at a lower fluid column H2 pressure than the PIP canister and so the flow of exhaled gas follows the path of least resistance through second valve 312 and the PEEP canister 362 to the environment. A humidification device, filters, or a temperature control device may be optionally employed in system 30. As stated before, system 30 may include other components described herein but not shown in these exemplary simplified illustrations for simplification (e.g., filters, temperature controllers, and so on).

Each of the gas flow control valves (310, 312) may be controllable using any suitable operating means that can controllably shut and open said valves during the inspiration and expiration phases, respectively. In one non-limiting embodiment, the valves are electrically operated by solenoids (solenoid valves) that receive a control signal from a pressure sensor or a controller to energize respective solenoids to pull or push a magnetic plunger and cause the valves to controllably open or shut as needed during the breathing cycle. In another non-limiting embodiment, the valves comprise pinch valves that have respective actuators which relax or constrict to allow or to prevent gas flow therethrough. Other valve designs may be used as known to those skilled in the art. The valves may be controlled using a controller circuit, which may be implemented in a standalone compact form or may be a sub-unit in a greater processing or control circuit board. Raspberry pi, ASIC, off-the-shelf circuits or customer circuitry may be implemented on a printed circuit board, bread board or other form factor which takes an input, performs any required signal processing thereon, and delivers an output signal that actuates the shutoff valve preferably in a desired phase with the breathing cycle. The valve controller may be built into a body of the valve as shown in the exemplary and non-limiting drawing here or may be located externally to the body of the valve itself. In various embodiments, the present system is designed to be as compact, lightweight and cost efficient or rugged as necessary to meet the needs for its intended use.

In an aspect, a pressure sensor, flow sensor or combination flow and pressure sensing device is placed in fluid communication with the gas manifold as discussed herein. The flow sensor can measure tidal volumes as known to those skilled in the art. The sensor(s) can be used to provide an input signal to a processor or controller that controls the operation (opening/closing) of the shutoff valve with respect to the breathing cycle.

The first and second controllable valves described herein are gas isolation valves and are associated with the alternating inspiration and expiration phases of operation of the system.

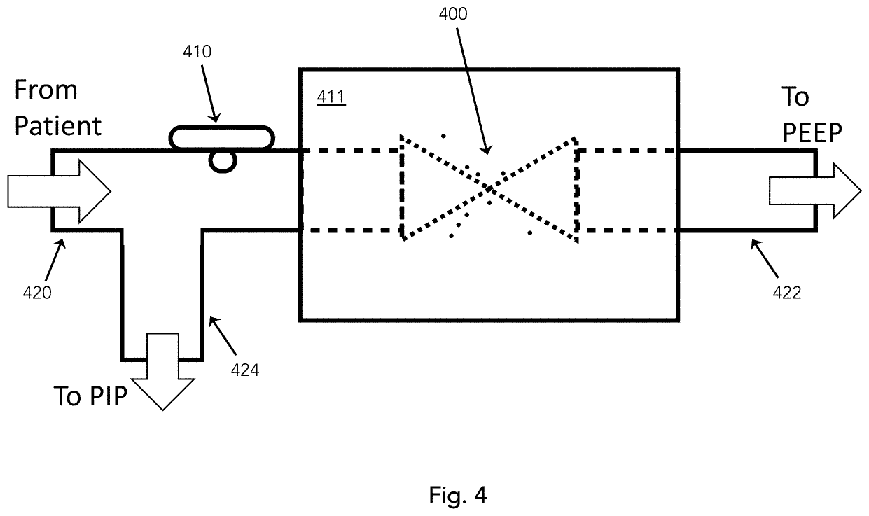

FIG. 4 shows an exemplary valve 400 with a sensor 410, that may comprise a pressure and/or gas flow sensor, disposed at the inlet port 420 and/or outlet port to the PEEP line 422 or to the PIP line 424 thereof. The sensor(s) 410 may be employed to provide an input to a processor circuit used to assist with patient breathing synchrony, and specifically in some embodiments for controlling the timing of the operation (opening, closing) of the gas control valves, or other controls operations, and for logging data, data reporting, or alarm operation in optional aspects. Optional alarms can indicate high/low pressure conditions at one or more points, high/low gas flow conditions, temperature conditions or other sensed parameters. In one example, an alarm may indicate an apnea condition. A valve unit assembly or valve block 411 contains the valve 400 and optionally other components related to the control or operation of valve 400. For example, optional embodiments may comprise a controls circuit, user interface, alarm circuits or other ancillary components.

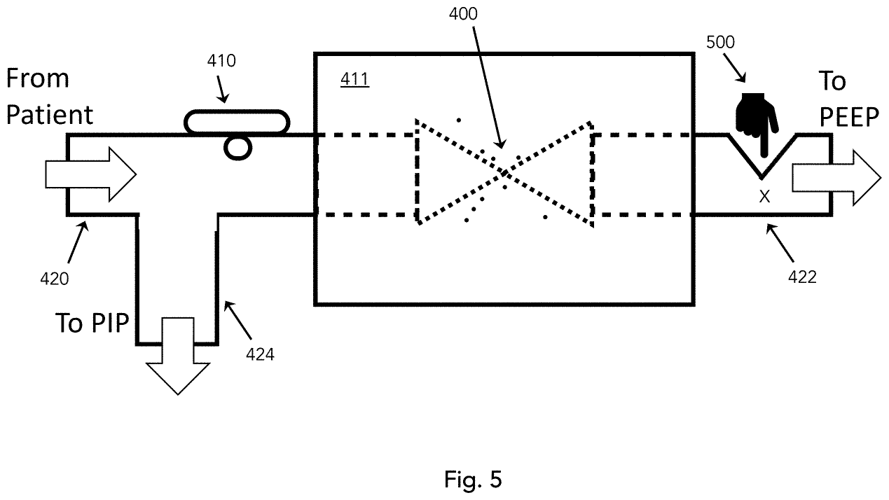

Manual operation of valve 400 may be implemented in certain optional and non-limiting instances. For example, capnography, pulse oximetry, physical manometry or other manual interventions and operations may be executed as suggested in FIG. 5 by visualization of bubbling action in the canisters of the present system using manual compression 500 which stops the flow of gas at “X”.

The present system and method may support operation in one or more modes including a mandatory, pressure assist and an apnea timer mode. However, the present system and method can be applied to any mode of operation for breathing assistance. In one mode, the system may be used for patients who are unable to breathe on their own because they are paralyzed or so weak that they cannot move their lungs to inhale/exhale on their own. In this case the system can apply as provided above a pressure to inflate and deflate the lungs for a patient by the force of the gas pressure provided to the patient, especially if the patient is equipped with an airtight nose and mouth mask. In this mode of operation, the system including a processor having a timer may be programmed or set to cause the necessary pressures at the patient's lungs to artificially cause the inspiration/expiration action. The system will be set to cause this cycle based on a timer, e.g., a determined number of seconds for each phase of the breathing cycle. In another mode, the system can be used with patients that are nominally able to move their lungs to inhale and exhale on their own, despite their underlying trauma or distressed condition requiring the present invention. In this case, as will be illustrated below, the system can use the pressure and/or gas flow sensors to synchronize or control the operation of the inspiration/expiration phases according to the patient's natural needs.

The present system may include optional electronic or processor-controlled user interfaces and other input/output means. For example, an optional display panel or touch-screen interface is possible to show output data and status conditions as well as to accept operator input commands or other setpoint program values.

Optionally, and in an aspect, the present system may be coupled to a commercial style ventilator system or part thereof so as to measure certain parameters, determine performance metrics or for other instrument or control purposes. Other input/output (I/O) accessories such as keypads and user interface sensors and visual or audible alarm units may be further configured and coupled to a processor or controller circuit of the invention to achieve these objectives.

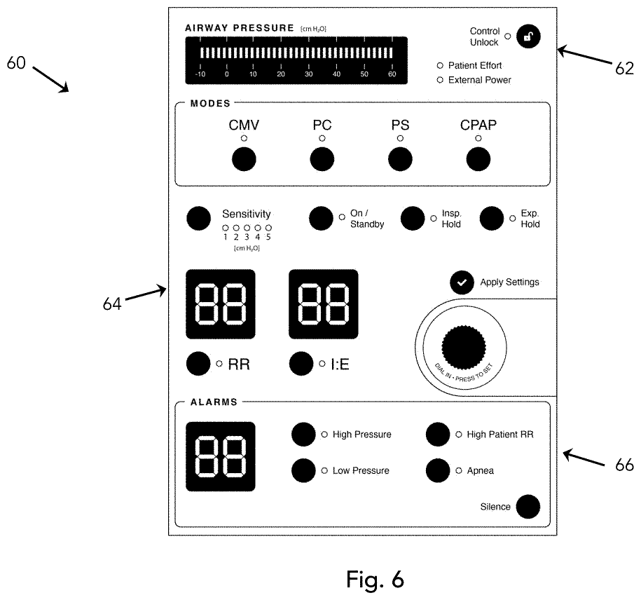

FIG. 6 illustrates an exemplary representation of an interface panel 60 that may aid the operation of the present system, but which is not required, and which can be partially or differently implemented according to specific needs. It is noted that the user interface and/or control panels of the present system may be integrated into the hardware of the system itself and locally thereto or may be implemented in a separate device (e.g., a smart phone or tablet or computer) in data communication with the system, or even remotely through some data communication link. Where the system does not require its own display and control panels it is possible to reduce the manufacturing complexity and cost of the system itself by using an operator's device or computer as the system's interface or control/display panel using suitably programmed applications or ‘apps’ on said operator device.

The exemplary interface 60 shown includes airway pressure output provided by a series of light emitting diodes (LED) bars 62, and can show the maximum, minimum and current values. Also, a controls section 64 and an alarms section 66 of the interface panel or display is possible, and can be configured in many ways as understood by those skilled in the art without limitation.

The present method and system may be designed to operate in a range of conditions and modes. In an aspect, the system can support a respiratory rate (RR) between 0 and 60 breaths per minute. In other aspects, the inspiratory time or duty cycle can range from 0% to 100% of a breath cycle as appropriate. In yet another aspect, the multiple modes of operation or use cases include: pressure control; pressure support; or CPAP mode. In still another aspect, the system can support operation using any one or more gases, including air, oxygen or a blend thereof, and may take a source pressure of 3 or more PSI. These examples are non-limiting and those skilled in the art will appreciate that variations and equivalents thereto are possible and depend on a desired application.

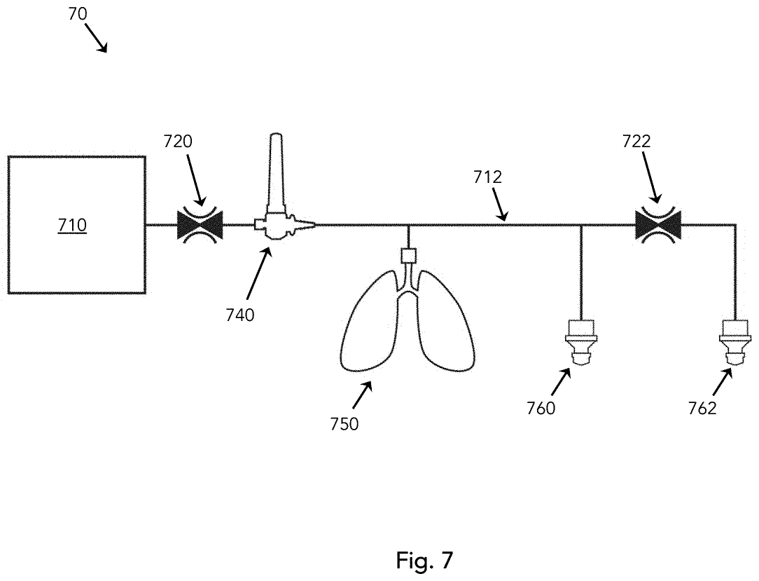

FIG. 7 illustrates an embodiment 70 having a breathing gas source or blender 710 flowing into a gas flow network 712 and pressure controlled by a pressure regulator 740. Here, the liquid containing PEEP and PIP reservoirs of the previous embodiments (which acted to control the pressure sensed at the open end of the respective submerged gas tubes) are replaced with respective back-pressure regulators 760, 762. Sometimes, and in an embodiment, these back-pressure regulating components are referred to as “PEEP valves” or “pop-off valves” in the field, but it is to be understood that one or both of the PIP and the PEEP gas lines may be terminated in or coupled to such pressure regulating valves 760, 762 as shown to establish adjustable gas pressures in the respective gas lines for inspiration and expiration phase control. The backpressure of the first such back-pressure regulation valve 760 is equivalent to the HP1 pressure discussed above, and the backpressure of the second such back-pressure regulation valve 762 is equivalent to the HP2 pressure discussed above, where HP1 is generally greater than HP2, and wherein the alternating opening and closing of gas control valves 720 and 722 causes respective intake of breathing gas from source 710 to the patient breathing interface 750 on the inspiration phase of operation, and causes exhalation of breathed gas from the patient breathing interface out of the second back-pressure regulation valve 762 on the expiration phase of operation of the system 70. As before, the indications and controls for the parts of the system, and the ability to add filters, humidifiers, temperature controls, etc., apply to this embodiment as well.

Another aspect of some embodiments of the invention allows for flushing the gas tubing or lines even after the patient exhales into them. For example, it may not be desirable for the sick patient to exhale into the portion of the gas lines upstream of the patient because upon inspiration the patient will be inhaling a slug of used or contaminated gas that has just come out of the patient's lungs on the previous exhalation. Therefore, in some optional embodiments, the upstream inspiratory Valve 1 may be left slightly open or fractionally open even in its “shut” state so as to substantially shut Valve 1 but to leave it slightly permeable to some clean gas flow from the source into the downstream parts of the system during such phases of the cycle.

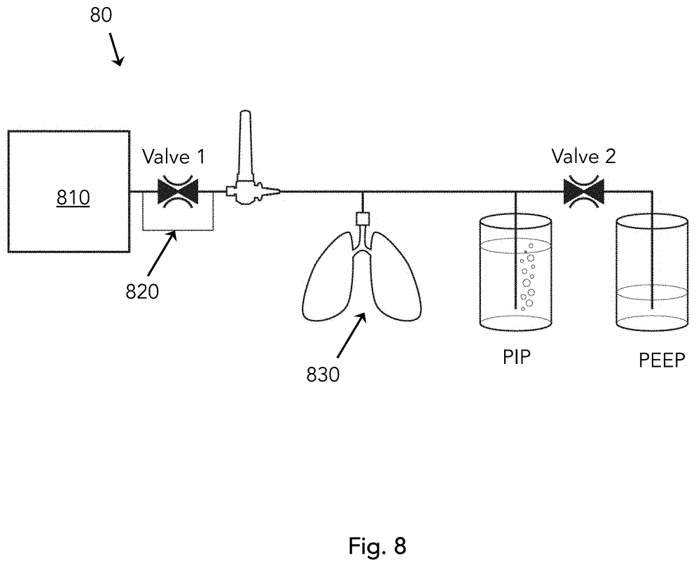

FIG. 8 illustrates an exemplary embodiment 80 where the above problem is addressed without the need to have Valve 1 partially open when in its “shut” or closed state. Instead, a small gas bypass line 820 around the upstream inspiratory valve (Valve 1) is provided. This bypass line 820 acts in an optional aspect to provide a fractional gas flow from the breathing gas source 810 into the gas lines or tubes upstream of the patient 830. This can act to provide a fractional leakage flow of fresh breathing gas into the system even when Valve 1 is shut and avoids the lines being filled with stagnant, used or contaminated gas during some phases of the cycle. The bypass line 820 also provides one-way gas flow to prevent rebreathing of exhaled gas. In an embodiment, a one-way check valve is installed in the gas flow network to direct gas flow only from the gas source or blender 810 to the other components of the system and to avoid back-flow of gas from a sick patient towards the gas source 810.

As mentioned earlier, the present system may be designed and configured to be compact, efficient and cost-effective compared to many other ventilator systems. The present system can be used for any ventilator mode(s) of operation, including those employing patient effort (patient's own muscle response to inhale and exhale).

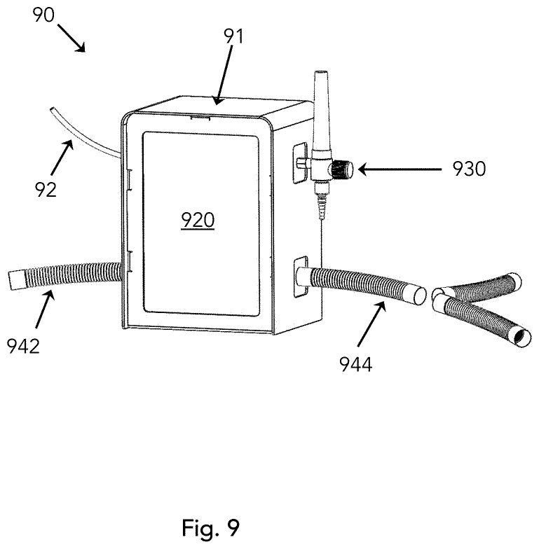

FIG. 9 illustrates an exemplary compact valve manifold 90 for use herewith whereby all or many of the major components of the system are disposed in a modular integrated housing design or block 91. The arrangement shown is only for illustration, and those skilled in the art will understand that numerous variations and configurations are possible without loss of generality. The breathing gas source and supply and control valves, e.g., valves Valve 1 and Valve 2 described herein, and regulator 930 described before, and other user interface 920 and gas management components, may be integrated into a same or connected modular block unit 90 which may comprise a gas manifold coupling to the gas network lines 942, 944 etc. Electrical power or control signals may be run into the unit 90 through one or more electrical/electronic connection lines 92. The unit 90 is compact, space and weight-efficient and relatively easy to manufacture and deploy.

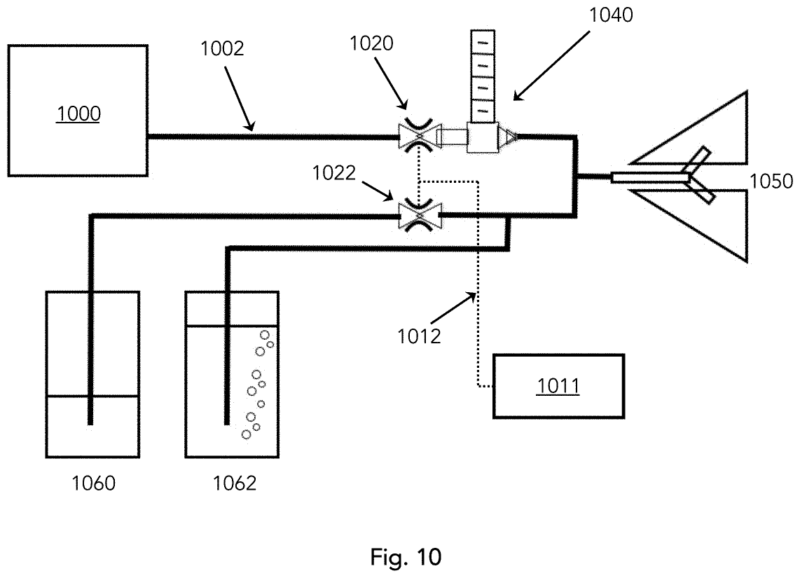

FIG. 10 illustrates a schematic representation of how a modular integrated housing as described above might include the inspiration and expiration valves 1020, 1022 for the two-valve design within the same housing, optionally along with other gas flow, control or measurement components. The topology of the gas lines 1002 leading from source 1000 to patient breathing interface 1050, regulator 1040, leading to PIP and PEEP canisters 1062, 1060 can be altered to meet the requirements of a given mechanical configuration of the system. For example, in some topologies, both valves can be housed in a same common housing block or unit as described herein.

Additionally, a valve controller 1011, comprising electrical or electronic circuits and/or machine-readable instructions executed thereon may be used to control the operation of gas isolation valves 1020 (Valve 1) and 1022 (Valve 2) during the biphasic operation of the system of this or other embodiments. That is, where valves Valve 1 and Valve 2 of the present invention are controllable to open and shut, any such embodiment may further comprise such a valve controller 1011, even where not illustrated in the accompanying simplified diagrams. For example, if valves 1020 (Valve 1) and/or 1022 (Valve 2) are electrically operated solenoid valves, electrical control signals may be respectively provided over control lines (or bus) 1012 from controller 1011 to the valve solenoids to operate them.

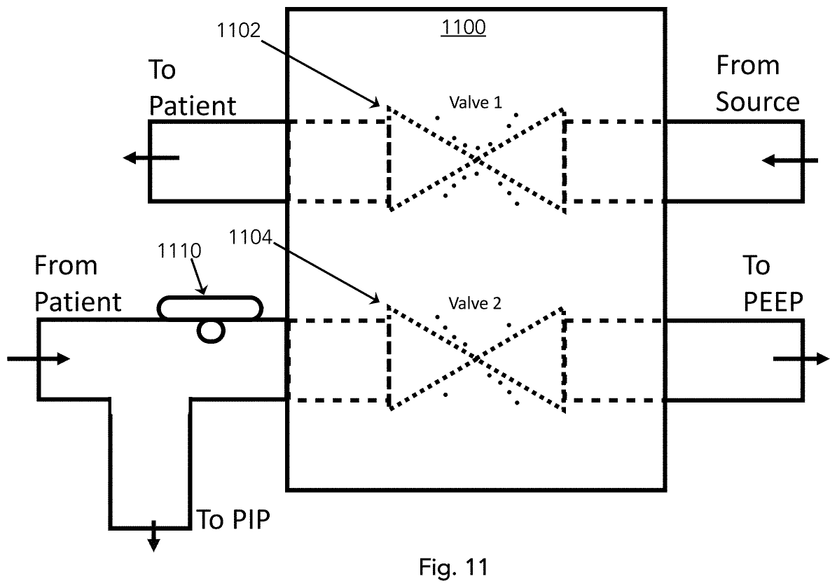

FIG. 11 illustrates a schematic representation of the two valves for inspiration 1102 and expiration 1104, sometimes configured and arranged in a common valve block or housing 1100, as described herein, in their relative placement within the modular integrated housing 1100 of some non-limiting embodiments. The valve system can employ one or more sensors 1110 as described earlier, which may be used to control the operation (opening, shutting, throttling) of one or more of Valve 1 and Valve 2.

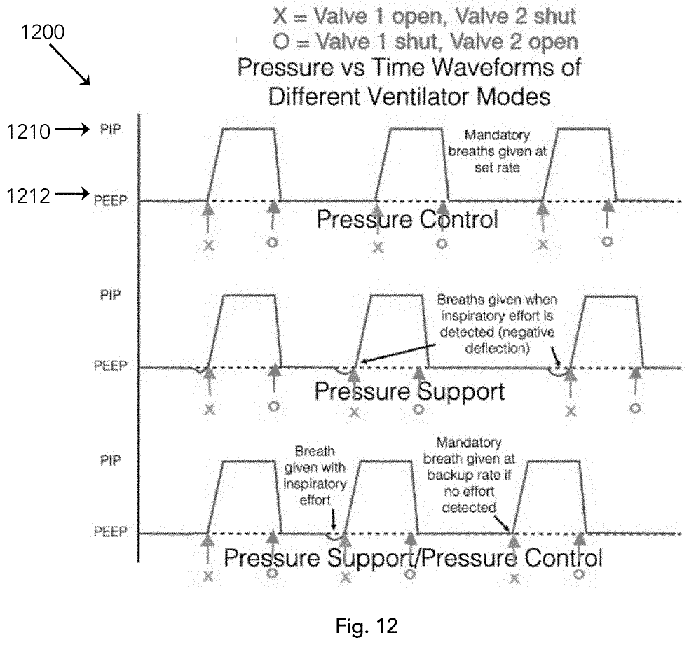

FIG. 12 illustrates exemplary timing waveforms 1200 showing the breathing cycle in various modes of operation of the present system. The PIP 1210 and PEEP 1212 cycles are shown and the relative open/shut operation of the inspiration (Valve 1) and expiration (Valve 2) valves are illustrated in relation to the same. In a pressure control mode of operation, mandatory breaths are given at a set rate or periodicity, which may be selected or entered into a user interface by the medical staff. In a pressure support mode, breaths are given when inspiratory effort is detected at the negative deflection points in the breathing cycle. In a pressure support/pressure control combined mode, breaths are given with inspiratory effort at the shown times and mandatory breaths are given at a backup rate if no effort by the patient is detected. The relative times at which the inspiratory valve (Valve 1) and expiratory valve (Valve 2) open and shut under system control are provided in the timing diagram for the operating conditions described.

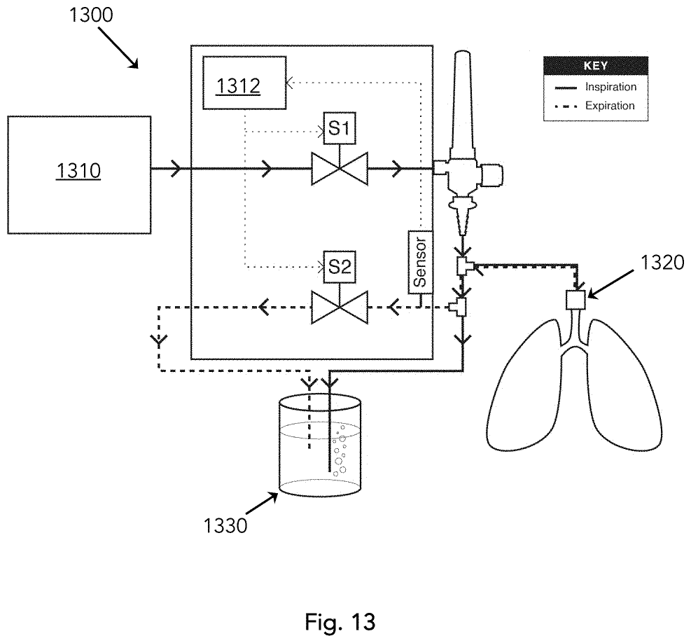

FIG. 13 illustrates another exemplary embodiment 1300 of the invention including a breathing gas source or blender 1310 providing gas to a patient breathing interface and patient 1320, a PIP/PEEP fluid back-pressure regulating canister 1330, and controlled by a controller 1312 that is arranged and adapted to control the functioning of at least first and second gas control valves S1 and S2 and other aspects. The figure also highlights the inspiration flow path of the gases in a first line shape (solid lines) and the expiration flow path of the gases in a second line shape (dashed lines).

In some optional aspects, the system is coupled to a data communication network, which can directly or indirectly exchange information over the Internet or another wider area network, a local hospital network and so on. The system may be fully or partially remotely controlled using remote to local interfaces, and/or the system may transmit data for telemedicine applications, for general data logging and collection, analysis or other purposes. The data from the system may be associated with a serial number or unique identifier of the system so that a central command center can associate the data with a given patient, clinic, location or other parameter.

Optionally, the system may be substantially encased in a housing that contains and protects the control circuits, processors, or even the valve and sensor units so that the system only needs external gas line connections, e.g., Tygon or other tubing to connect to the patient interface and to the gas supply and the canister units.

The system housing or an external user interface may be equipped with electronic or other interface units that accept an input from an operator or clinician for example to set the desired inspiratory time or the respiration rate. A numerical touch pad or similar interface unit could be used to achieve this function but is not required for the basic operation of the system.

A smart phone or personal communication device interface may be incorporated so that the above user interface, display, and touch screen inputs are provided by the stand-alone phone unit as opposed to being built into the system itself. In this way, the system is modular and can reduce the system cost and allow an operator to use their device as a user interface, data logger, network communication interface for exchanging data into or out of the system to a greater network of machines and cloud-based data stores, servers, central command centers, etc. By coupling the basic system to such external devices, interfaces and servers, programmable alarms and status indicators and data trackers and remote adjustment and control can be employed as would be appreciated upon review of this disclosure.

The present invention also provides a method for delivering breathing gas to a patient in need of assisted breathing during an inspiration phase, and for taking exhaust gas from the patient during an expiration phase. The gas delivered to and from the patient is exchanged by way of a patient breathing interface as described herein. In a first (inspiration) phase of operation, a first gas isolation valve in the upstream section of the gas flow network is opened so as to release or deliver breathing gas from a source thereof to the gas flow network and to the patient breathing interface. In a second (expiration) phase of operation, the first gas isolation valve is shut or closed (e.g., using a solenoid or other controllable valve action) and a second gas isolation valve downstream of the patient breathing interface is opened. The second gas isolation valve thereby permitting exhaust of used gas from the patient breathing interface to a second fluid reservoir by way of a second gas tube submerged to a second depth below a fluid surface as described. In an aspect, the first and second gas isolation valves are operated substantially out of phase with one another (when one is open the other is closed). The method thus controllably blocks flow of gas through portions of the gas flow network as described to achieve an assisted breathing cycle of inspiration and expiration whether solely driven by the system pressures and/or driven by the patient's lungs and muscular action, or a combination thereof. Describing a gas isolation valve as open means that it can substantially pass gas therethrough, while describing a gas isolation valve as closed or shut means that the valve substantially blocks or prevents flow therethrough, although valves may have restrictions when open and leakage when closed or shut as understood by those skilled in the art.

Those skilled in the art will appreciate that the two-valve designs provided in various embodiments can isolate or substantially shut breathing gas flow from a breathing gas source to the gas network when not needed, during expiration phases of the biphasic system's operation. This solves the problem found in some prior systems where breathing gas escapes through the gas flow network into the environment and is not used by the patient during some phases of operation.

The present disclosure should not be considered limited to the particular embodiments described above. Various modifications, equivalent processes, as well as numerous structures to which the technology may be applicable, will be apparent to those skilled in the art to which the technology is directed upon review of this disclosure.

What is claimed is:

1. A biphasic breathing assistance system, comprising:
a breathing gas source comprising at least one gas to be used for breathing by a patient using said system;
a gas flow network coupled to said breathing gas source;
a patient breathing interface coupled to said gas flow network, configured and arranged to move an amount of the breathing gas to the patient during a first (inspiration) phase of operation and to take an amount of exhaled gas from the patient during a second (expiration) phase of operation;
a first (PIP) gas line coupled to the gas flow network at a first end of the first (PIP) gas line and coupled to a first back-pressure regulator having a first back-pressure setting at a second end of the first (PIP) gas line;
a second (PEEP) gas line coupled to the gas flow network at a first end of the second (PEEP) gas line and coupled to a second back-pressure regulator having a second back-pressure setting at a second end of the second (PEEP) gas line;
a first gas isolation valve disposed in the gas flow network downstream of the breathing gas source and upstream of the patient breathing interface;
a second gas isolation valve disposed in the gas flow network between said first (PIP) gas line and said second (PEEP) gas line; and
wherein said first gas isolation valve is configured and arranged to be open during said first (inspiration) phase of operation of the system while the second gas isolation valve is shut, and the first gas isolation valve is configured and arranged to be shut during said second (expiration) phase of operation of the system while the second gas isolation valve is open.

2. The system of claim 1, further comprising a valve controller, electrically coupled to said first and second gas isolation valves, that controls the operation of said first and second gas isolation valves, enabling the system to alternately open and shut said valves, respectively, during its biphasic operation.

3. The system of claim 1, wherein the first back-pressure regulator comprises a first liquid reservoir containing a first liquid volume, and a first submerged height (H1) of said first (PIP) gas line below a surface of said first liquid volume so as to provide a first hydrostatic pressure.

4. The system of claim 3, wherein the second back-pressure regulator comprises a second liquid reservoir containing a second liquid volume, and a second submerged height (H2) of said second (PEEP) gas line below a surface of said second liquid volume so as to provide a second hydrostatic pressure.

5. The system of claim 4, wherein said first submerged height (H1) is greater than said second submerged height (H2) and said first hydrostatic pressure is greater than said second hydrostatic pressure.

6. The system of claim 1, said first and second back-pressure regulators comprising a common shared liquid reservoir containing a liquid volume, the first back-pressure regulator further comprising a first (PIP) gas line submerged below a surface of the liquid volume to a first submerged height (H1) and the second back-pressure regulator further comprising a second (PEEP) gas line submerged below the surface of the liquid volume to a second submerged height (H2), wherein H1 is greater than H2).

7. The system of claim 1, wherein said first and second back-pressure regulators comprise respective first and second pop-off valves, each set to a given pressure setting wherein the back-pressure provided by the first pop-off valve is greater than the back-pressure provided by the second pop-off valve.

8. The system of claim 1, further comprising a humidifier inline with said gas flow network to controllably adjust a humidification level of gas provided to said patient breathing interface.

9. The system of claim 1, further comprising a temperature control unit inline with said gas flow network to controllably adjust a temperature of gas provided to said patient breathing interface.

10. The system of claim 1, further comprising a pressure regulator downstream of said breathing gas source that controls a gas pressure in said gas flow network.

11. The system of claim 1, wherein at least said first and second gas isolation valves are disposed in a common housing valve block containing a gas manifold.

12. The system of claim 1, further comprising a gas bypass line configured and arranged to provide continuous gas flow from the breathing gas source to other components of the gas flow network.

13. A method for assisting patient breathing using a biphasic breathing assistance apparatus, the method comprising:
providing a pressure-regulated breathing gas source to a supply side of a gas flow network;
during an inspiration phase, opening a first gas isolation valve between said breathing gas source and a patient breathing interface coupled to said gas flow network to permit flow of breathing gas from said breathing gas source to said patient breathing interface, and closing a second gas isolation valve between said patient breathing interface and a gas exhaust side of said gas flow network;
during an expiration phase, closing said first gas isolation valve and opening said second gas isolation valve so as to substantially block flow of breathing gas to the patient breathing network but to permit flow of exhaled gas from said patient breathing network to said gas exhaust side of the gas flow network.

14. The method of claim 13, further comprising controllably setting a first hydrostatic pressure at the patient breathing interface during the inspiration phase and a second hydrostatic pressure at the patient breathing interface during the expiration phase by controllably coupling the patient breathing interface to first and second immersed gas tubes, respectively during the inspiration and expiration phases.

15. The method of claim 13, further comprising controllably setting a first gas pressure at the patient breathing interface during the inspiration phase and a second gas pressure at the patient breathing interface during the expiration phase by controllably coupling the patient breathing interface to first and second calibrated pop-off valves, respectively during the inspiration and expiration phases.

16. A system providing breathing gas to a patient, comprising:
a breathing gas source coupled to and in fluid communication with a gas flow network;
a patient breathing interface in fluid communication with said gas flow network;
a first liquid reservoir, containing a first volume of liquid, the first liquid reservoir in fluid communication with said gas flow network through a first gas tube having a first end thereof coupled to said gas flow network and a second end thereof submerged at a first depth defining a first liquid column height H1 and a corresponding first hydrostatic pressure HP1;
a second liquid reservoir, containing a second volume of liquid, the second liquid reservoir in fluid communication with said gas flow network through a second gas tube having a first end thereof coupled to said gas flow network and a second end thereof submerged at a second depth defining a second liquid column height H2 and a corresponding second hydrostatic pressure HP2;
a first gas isolation valve disposed in said gas flow network between the breathing gas source and the patient breathing interface, the first gas isolation valve having a first (open) state to permit flow of said breathing gas from the breathing gas source to the patient breathing interface during an inspiration phase of operation of the system, and a second (closed) state to block flow of said breathing gas from the breathing gas source to the patient breathing interface during an expiration phase of operation of the system; and
a second gas isolation valve disposed in said gas flow network between the first ends of said first and second gas tubes, the second gas isolation valve having a first (open) state to permit flow of exhaled gas from the patient breathing interface to the second liquid reservoir during the expiration phase of operation of the system, and a second (closed) state to block flow of exhaled gas from said patient breathing interface to said second liquid reservoir.
